(19) United States
(12) Patent Application Publication
(10) Pub. No.: US 2017/0073721 A1
(43) Pub. Date: Mar. 16, 2017

(54) TESTING METHODS AND APPARATUS

(71) Applicant: Bactest Limited, Cambridge (GB)

(72) Inventors: Toby William RICKETT, Cambridge (GB); Derek PRICE, Cambridge (GB); Annie BROOKING, Cambridge (GB)

(21) Appl. No.: 15/124,691

(22) PCT Filed: Mar. 9, 2015
(86) PCT No.: PCT/GB2015/050677

§ 371 Date: Sep. 9, 2016

(30) Foreign Application Priority Data
Mar. 10, 2014 (GB) 1404196.6
Mar. 20, 2014 (GB) 1405010.8

(51) Int. Cl. C12Q 1/04; C12M 1/00; C12M 1/12; C12M 1/34

(52) U.S. Cl. CPC C12Q 1/04; C12M 41/46; C12M 23/34; C12M 41/12; C12M 41/40; C12Q 1/045; C12M 37/00

(57) ABSTRACT

We describe a method of selectively testing a liquid sample for the presence of a specific living biological entity, the method comprising: preparing a plurality of culture vessels each for selective detection of the presence of a different living biological entity, wherein preparing a said culture vessel comprises: providing a culture vessel comprising a sealable chamber for receiving and culturing a liquid sample such that presence of said biological entity is detectable by detecting a change in pressure in a head space of said chamber; providing a selective growth medium in said culture vessel, wherein said selective growth medium is in a dry form and selectively facilitates growth of a specific living biological entity, and providing the culture vessel with an identifier to identity said specific biological entity; providing a test system comprising a device to receive said culture vessel and to detect said change in pressure in said head space of said chamber, the device further comprising a temperature control system, and a data processor to monitor said detected change in pressure, to control said temperature control system to regulate a temperature of a liquid sample in said culture vessel, and to signal detection of the presence of a biological entity in said culture vessel from said detected change in pressure; storing a plurality of entity—selective test protocols in a memory of said test system, wherein a said test protocol defines one or both of a temperature of incubation of said liquid sample in said culture vessel and a threshold change in pressure in said head space of said chamber for detection of the target entity, wherein said test protocols are respectively identified according to one of a plurality of specific biological entities for which they selectively facilitate growth; selecting, from said plurality of culture vessels, a culture vessel to selectively detect a target said specific biological entity; providing a test liquid sample to said chamber of said selected culture vessel; using the identifier on said elected culture vessel to select a corresponding test protocol for said target specific biological entity; and running said selected test protocol on said test system to incubate said test liquid sample according to said test protocol to determine whether said target specific biological entity is present within said test liquid sample; wherein said determination comprises selection dependent upon both said selective growth medium and said entity-selective test protocol.

### FIELD OF THE INVENTION

This invention relates to methods and apparatus for selectively testing liquid samples for the presence of specific living biological entities.

### BACKGROUND TO THE INVENTION

We have previously described a system for monitoring the metabolism/growth of microorganisms, the system comprising a sealed chamber with a flexible diaphragm to provide sensitive measurements of variations of gas pressure in the headspace above a culture liquid. For background, reference may be made, for example, to U.S. Pat. No. 8,389,274.

FIGS. 1a and 1b show, schematically, an embodiment of a similar device 100 under, respectively, normal atmospheric pressure and negative pressure (in operation either negative pressure or positive pressure may be produced). Thus a culture 102 of biological material undergoes metabolism and growth during which it exchanges gases with the aqueous liquid (water) carrying cells depending upon various factors gas may be used and/or produced, for example the cells may produce carbon dioxide during respiration. A gaseous headspace 104 of the sealed culture chamber 106 thus experiences changes in pressure due to exchange of gas with the culture medium, and these are monitored by a diaphragm 108 and converted to an electronic pressure signal 110 which may, for example, be digitised and processed electronically by hardware, software or a combination of the two. Preferably the system also includes an agitator 112 and temperature control (not shown), as well as a sealable inlet/outlet port 114.

FIG. 2 shows a physical embodiment of a test device 200 with 2 chambers 202, 204, each configured to accept a culture vessel 100 of the general type shown in FIG. 1. Optionally, though not essentially, one of these may act as a control. Each chamber provides a magnetic drive for agitator 112 and a temperature control system to allow the temperature in each culture vessel to be regulated, in embodiments in the range 14 to 44 degrees or 10 to 60 degrees. The device also has a user interface 206 comprising a display and user controls, and communications such as a USB connection and/or wired or wireless network connection. In preferred embodiments the system can be used independently or connected to a separate computer; the external connection facilitates, but is not necessary for, control of the system. For example a quality assurance manager may design and download protocols for the system and upload test results for analysis and/or audit. Connection to an external computer system facilitates downloading a custom test protocol to the device. In embodiments the system also has a removable Flash™ memory card for storing experimental results and/or data defining test protocols. Preferred embodiments of the device are relatively lightweight and portable and are powered by a low voltage dc source.

In use samples are introduced into the two culture vessels and the system controls the growth conditions and logs changes in pressure. As previously described and shown in FIG. 1 the pressure sensing in embodiments uses a non-invasive process which isolates the sensors and, importantly, provides a barrier to protect both the biological culture and the operator.

The system operates as a sensitive microbial respirometer, detecting metabolic activity by measuring pressure transients relating to gaseous exchanges within the closed culture vessel (which in embodiments has a volume in the range 10 ml-100 ml) as a result of microbial respiration. The mixing helps to homogenise the culture and also facilitates gaseous exchange which is important to convert the effects of metabolic processes into detectable pressure transients (which may be positive or negative).

Whilst embodiments of the above described system are very effective at detecting the growth of bacteria and other living biological entities, in practice it is important to be able to accurately distinguish between the presence of different entities, for example different bacteria.

### SUMMARY OF THE INVENTION

According to the present invention there is therefore provided a method of selectively testing a liquid sample for the presence of a specific living biological entity, the method comprising: preparing a plurality of culture vessels each for selective detection of the presence of a different living biological entity, wherein preparing a said culture vessel comprises: providing a culture vessel comprising a sealable chamber for receiving and culturing a liquid sample such that presence of said biological entity is detectable by detecting a change in pressure in a headspace of said chamber; providing a selective growth medium in said culture vessel, wherein said selective growth medium is in a dry form and selectively facilitates growth of a specific living biological entity, and providing the culture vessel with an identifier to identity said specific biological entity; providing a test system comprising a device to receive said culture vessel and to detect said change in pressure in said headspace of said chamber, the device further comprising a temperature control system, and a data processor to monitor said detected change in pressure, to control said temperature control system to regulate a temperature of a liquid sample in said culture vessel, and to signal detection of the presence of a biological entity in said culture vessel from said detected change in pressure; storing a plurality of entity-selective test protocols in a memory of said test system, wherein a said test protocol defines one or both of a temperature of incubation of said liquid sample in said culture vessel and a threshold change in pressure in said headspace of said chamber for detection of the target entity, wherein said test protocols are respectively identified according to one of a plurality of specific biological entities for which they selectively facilitate growth; selecting, from said plurality of culture vessels, a culture vessel to selectively detect a target said specific biological entity; providing a test liquid sample to said chamber of said selected culture vessel; using the identifier on said selected culture vessel to select a corresponding test protocol for said target specific biological entity; and running said selected test protocol on said test system to incubate said test liquid sample according to said test protocol to determine whether said target specific biological entity is present within said test liquid sample; wherein said determination comprises selection dependent upon both said selective growth medium and said entity-selective test protocol.

Broadly speaking, although the specificity provided by selecting a particular growth medium is not, on its own, sufficient to selectively grow a particular strain of bacteria, and although selection of a particular test protocol, by itself, may not confer adequate selectivity, experiments have established that the combination of a selective growth media with a defined test protocol can very effectively selectively grow a specific type of biological entity such as a particular species of bacteria. This selective detection of a specific target entity, such as a specific species of bacteria, can be achieved by embodiments of the above described method which link a particular growth medium to a particular test protocol, for example test temperature and/or a threshold (positive or negative) change in pressure. Optionally a test protocol may include other parameters, for example a test time (for example a duration or minimum duration or a detection time window) and/or a colorimetric measurement and/or test sample mixing (for example defining a proportion of time for which the sample is mixed). In general, cultures of different entities may produce colour changes in the medium that are indicative of certain metabolic processes of that entity, and this may form part of the identification process, in combination with the test protocol (incubation temperature and/or pressure change) and the specific growth medium.

In some preferred embodiments the test protocol defines both the temperature of incubation of the liquid sample and the threshold change in pressure for detection of the specific, target entity. As described later, this threshold pressure may be either positive or negative (or even a combination of the two, for example a defined fall followed by a defined rise, or vice-versa). The definition of a specific threshold pressure has been found to be important in distinguishing a relevant signal from the background. The pressure may be defined, for example, in terms of a number of millibars change in pressure.

In embodiments the culture vessel is sealed by a liquid-impermeable septum through which the liquid sample can be injected into the culture vessel (such an arrangement is, by its nature, self-sealing). This facilitates maintenance of a controlled environment within the culture vessel and minimises contamination. Alternatively a sample port with a removable cap may be employed for adding the sample and afterwards inhibiting contamination.

In embodiments the selective growth medium may be combined with a selective growth inhibiting substance to selectively inhibit growth of entities other than the specific, target entity. In embodiments the growth inhibiting substance may comprise a particular antibiotic or mixture of antibiotics and/or a chemical substance which selectively affects growth and/or a mixture of one or more such chemical substances optionally with one or more antibiotics. Advantageously the growth medium and/or growth inhibiting substance are provided in particulate form within a water-soluble capsule housing, to maintain sterile conditions whilst quickly releasing the growth and/or growth inhibiting substances when needed. Although gelatin may be used for the capsule housing, preferred materials employ starch or cellulose, for example HPMC (hydroxypropyl methylcellulose).

One difficulty with using selective growth inhibiting substances such as antibiotics is that they are often heat labile, and thus it is difficult to sterilise the growth medium-antibiotic combination. In embodiments, therefore, after the growth medium and growth inhibiting substance are combined the combination is sterilised by irradiation with gamma rays.

Surprisingly, not only can tests of the type describe above selectively detect the presence of specific target entities, extensive experiments have also determined that they can accurately predict the number (count) of entities in the original liquid sample based on the time interval needed for a particular change in pressure. A graph of the logarithm of the number of entities (N) in the original liquid sample against time is substantially a straight line (for a particular entity/test protocol). The parameter(s) defining this line, in particular the slope of the line, may be determined by a calibration procedure, and because they are not machine-specific this calibration needs to be performed only once for each entity/test protocol. (In principle the calibration data may be defined by a curve rather than a (straight) line, again specified by one or more parameters, but this has not been found necessary in practice). The calibration data may then be stored, either locally on the device or in an associated computer system, and used to determine an estimated number of the target entities in the test liquid sample from the detected change in pressure. This may be done either by a single measurement or by fitting a line to succession of measurements.

Embodiments of the above described testing method may be used to detect specific bacteria and/or fungi (such as yeast and mould), and protozoa as well as other biological entities. Examples of bacteria species which may be selectively detected in this way include Escherichia coli, salmonella (in its various types), Listeria monocytogenes, and many others.

In a related aspect the invention provides a liquid sample test system for selectively testing a liquid sample for the presence of a specific living biological entity, the system comprising: a plurality of culture vessels each for selective detection of the presence of a different living biological entity, a said culture vessel comprising a sealable chamber for receiving and culturing a liquid sample such that presence of said biological entity is detectable by detecting a change in pressure in a headspace of said chamber, said culture vessel chamber including a selective growth medium in said culture vessel, wherein said selective growth medium is in a dry form and selectively facilitates growth of a specific living biological entity, said culture vessel having an identifier to identify said specific biological entity; a test system comprising a device to receive said culture vessel and to detect a change in pressure in said headspace of said chamber, the device further comprising a temperature control system, and a data processor to monitor said detected change in pressure, to control said temperature control system to regulate a temperature of a liquid sample in said culture vessel, and to signal detection of the presence of a biological entity in said culture vessel from said detected change in pressure; wherein said data processor comprises memory storing a plurality of entity-selective test protocols, wherein a said test protocol defines one or both of a temperature of incubation of said liquid sample in said culture vessel and a threshold change in pressure in said headspace of said chamber for detection of the target entity, wherein said test protocols are respectively identified according to one of a plurality of specific biological entities for which they selectively facilitate growth; and wherein the identifier on said selected culture vessel is usable to select a corresponding test protocol for said target specific biological entity; and wherein said selected test protocol is useable by said test system to incubate said test liquid sample according to said test protocol to determine whether said target specific biological entity is present within a test liquid sample.

The invention further provides processor control code and/or data for the above described data processor and, in embodiments, for estimating the number of entities in the original liquid sample. This code/data may be provided on a non-transitory data carrier such as a disc or programmed memory. Code and/or data to implement embodiments of the invention may comprise source, object or executable code in a conventional programming language; such code and/or data may be distributed between a plurality of coupled components in communication with one another.

In a further related aspect the invention provides a method of preparing a culture vessel, for selectively testing a liquid sample for the presence of a specific living biological entity, the method comprising: providing a culture vessel comprising a sealable chamber for receiving and culturing a liquid sample such that presence of said biological entity is detectable by detecting a change in pressure in a headspace of said chamber; providing a selective growth medium in said culture vessel, wherein said selective growth medium is in a dry form and selectively facilitates growth of a specific living biological entity; and providing the culture vessel with an identifier to identity said specific biological entity.

In a corresponding aspect the invention provides a culture vessel prepared by the method.

The invention also provides a set of culture vessels for selectively testing a liquid sample for the presence of a specific living biological entity, the set of culture vessels comprising a plurality of culture vessels each for selective detection of the presence of a different living biological entity, each said culture vessel comprising: a sealable chamber for receiving and culturing a liquid sample such that presence of said biological entity is detectable by detecting a change in pressure in a headspace of said chamber; a selective growth medium in said chamber, wherein said selective growth medium is in a dry form and selectively facilitates growth of a specific living biological entity; each said culture vessel having an identifier to identify said specific biological entity.

Surprisingly, a good estimation of the number of living biological entities originally present in a liquid sample may be made using a non-selective growth-medium, irrespective of the particular species of entity present. This is surprising because one might expect, for example, that different types of bacteria would grow/metabolise at different rates. Nonetheless it has been found that when a non-specific growth medium is employed, common calibration data may be used, the calibration data defining a relationship between time to a greater than threshold change (increase) in pressure and a logarithm of the number of entities initially present in the sample. (This line has a negative slope since the fewer the number of entities initially present the longer the time for a particular pressure change).

Thus in a further aspect the invention provides a liquid sample test system for testing a liquid sample for the presence of a living biological entity, the system comprising: a culture vessel comprising a sealable chamber for receiving and culturing a liquid sample such that presence of said biological entity is detectable by detecting a change in pressure in a headspace of said chamber, said culture vessel chamber including a growth medium for said living biological entity; and a test system comprising a device to receive said culture vessel and to detect said change in pressure of said headspace of said chamber, the device further comprising a temperature control system, and a data processor to monitor said detected change in pressure, to control said temperature control system to regulate a temperature of a liquid sample in said culture vessel, and to signal detection of the presence of a biological entity in said culture vessel from said detected change in pressure; wherein said data processor comprises memory storing calibration data defining a relationship between a time to a threshold said change in pressure and an initial number of said biological entities; and wherein said data processor comprises processor control code to determine an estimated number of said target specific biological entities in said test liquid sample from said detected change in pressure and said calibration data.

The invention also provides a corresponding method, and corresponding computer program code. In embodiments of this system/method the culture vessel holds a defined quantity of the growth medium, for example sufficient that the quantity of growth medium is not a growth-limiting factor, but not so much that the concentrations of the growth medium in solution is toxic to the growing entities.

In embodiments of the system the stored code includes codes to determine a numeric range for the estimated number of entities originally present dependent on statistical data for the defined relationship stored in the memory. Thus, in embodiments, the calibration data includes data defining an estimated standard deviation for the estimated number of entities, from which a numeric range, or equivalently error bars, can be determined (for example in the form +/−n standard deviations, where n is a factor, say in the range 1-3). Although it has been established that a good prediction of the estimated number of entities originally present can be made using non-specific growth medium, nonetheless in some preferred embodiments additionally or alternatively the calibration data defines a different log N˜time relationship for each of a plurality of different types of biological entities (for example bacterial species). The stored code may then include code to input data defining an expected (or selected) type of biological entity and the corresponding relationship can then be used to determine a more accurate numerical estimate and/or range for the original number of entities present in the test sample.

In embodiments the estimated number/range of entities (N) is defined in terms of CFUs (colony-forming units).

In a still further aspect the invention provides a method of testing an aqueous sample for a specific living biological entity, the method comprising: preparing a culture vessel comprising a sealable chamber for receiving and culturing a liquid sample such that growth of said biological entity is detectable by detecting a change in pressure in a headspace of said chamber; providing a dry-form selective growth medium for said biological entity in said chamber, and identifying the culture vessel; providing a test system for regulating a temperature of said aqueous sample and detecting a change in pressure in said headspace; storing a plurality of entity-selective test protocols in a memory of said test system, wherein a said test protocol defines one or both of a temperature of incubation of said aqueous sample and a threshold change in pressure in said headspace of said chamber for detection of said specific living biological entity, wherein said test protocols are identified according to the corresponding specific biological entities for which they facilitate selectively growth; providing an aqueous sample to said chamber of said culture vessel to rehydrate said growth medium; using the identifier on said selected culture vessel to select a corresponding test protocol for said target specific biological entity; and running a test protocol on said test system corresponding to a specific biological entity for which said growth medium is selective, incubating said sample according to said test protocol to determine whether said specific biological entity is present.

The invention further provides a liquid sample test system for testing a liquid sample for the presence of a specific living biological entity, the system comprising: a culture vessel comprising a sealable chamber for receiving and culturing a liquid sample such that presence of said biological entity is detectable by detecting a change in pressure in a headspace of said chamber, said culture vessel chamber including a selective growth medium for said specific living biological entity; and a test system comprising a device to receive said culture vessel and to detect said change in pressure of said headspace of said chamber, the device further comprising a temperature control system, and a data processor to monitor said detected change in pressure, to control said temperature control system to regulate a temperature of a liquid sample in said culture vessel, and to signal detection of the presence of said specific biological entity in said culture vessel from said detected change in pressure; wherein said data processor comprises memory storing test protocol data defining one or both of a temperature of incubation of said liquid sample and a threshold change in pressure in said headspace of said chamber for detection of said specific living biological entity; and wherein said data processor comprises processor control code to determine presence of said specific target specific biological entity in said test liquid sample from said detected change in pressure and said test protocol data.

In some preferred embodiments of the above described methods and systems living biological entities that have been incubated within the culture vessel are treated to render them non-viable, or at least non-culturable. In some preferred embodiments this is done by using the temperature regulation system to increase the temperature of the liquid in a culture vessel to greater than a threshold temperature for greater than a threshold time. For example, the liquid in the culture vessel may be heated to greater than 63° C. for greater than 30 minutes.

In general the higher the temperature the shorter the time needed, and vice-versa. The precise temperature used may depend on the entity but is preferably greater than 50° C., more preferably greater than 60° C. The skilled person will appreciate that in general (like pasteurization) not all the living entities will be killed, but that a sufficient number will be killed to render the treated liquid safe. Moreover because the liquid is contained within a sealed chamber, handling for disposal is facilitated.

In some embodiments the threshold temperature and/or threshold time may be defined as part of a test protocol, but more generally a temperature and time can be used which is appropriate for all the target entities (for example bacteria) which are being incubated.

The skilled person will further appreciate that the above techniques to facilitate safe disposal may be employed independently of culturing a specific target organism and/or of estimating the number of (any type of) organisms initially present in a liquid sample.

Thus in a further aspect the invention provides a method of selectively testing a liquid sample for the presence of a living biological entity, the method comprising: providing a culture vessel comprising a sealable chamber for receiving and culturing a liquid sample such that presence of said biological entity is detectable by detecting a change in pressure in a headspace of said chamber; providing a growth medium in said culture vessel, wherein said growth medium is in a dry form and facilitates growth of a living biological entity, and providing a test system comprising a device to receive said culture vessel and to detect said change in pressure in said headspace of said chamber, the device further comprising a temperature control system, and a data processor to monitor said detected change in pressure, to control said temperature control system to regulate a temperature of a liquid sample in said culture vessel, and to signal detection of the presence of a biological entity in said culture vessel from said detected change in pressure; providing a test liquid sample to said chamber of said selected culture vessel; incubating said test liquid sample to determine whether said target specific biological entity is present within said test liquid sample; and then treating said incubated test liquid sample to render biological entities within said culture vessel non-viable or non-culturable.

In a related further aspect the invention provides a liquid sample test system for selectively testing a liquid sample for the presence of a living biological entity, the system comprising: a device to receive a culture vessel and to detect a change in pressure in said headspace of said chamber, said culture vessel comprising a sealable chamber for receiving and culturing a liquid sample such that presence of said biological entity is detectable by detecting a change in pressure in a headspace of said chamber; a temperature control system; and a data processor to monitor a detected change in pressure of said headspace, to control said temperature control system to regulate a temperature of a liquid sample in said culture vessel according to a test protocol, and to signal detection of the presence of a biological entity in said culture vessel from said detected change in pressure; wherein said data processor is configured to: incubate a test liquid sample to determine whether a biological entity is present within said test liquid sample; and then raise a temperature of said incubated test liquid sample to greater than a threshold temperature for greater than a threshold time to render biological entities within said culture vessel non-viable or non-culturable.

The invention also provides, separately, the (test) device) and the culture vessel of the above described liquid sample test system.

The skilled person will appreciate that this aspect of the invention may include the previously described features of embodiments/aspects of the invention to provide additional advantages.

### BRIEF DESCRIPTION OF THE DRAWINGS

These and other aspects of the invention will now be further described, by way of example only, with reference to the accompanying Figures in which:

FIGS. 1a and 1b show a culture vessel for use in embodiments of the invention under, respectively, normal atmospheric pressure and reduced pressure;

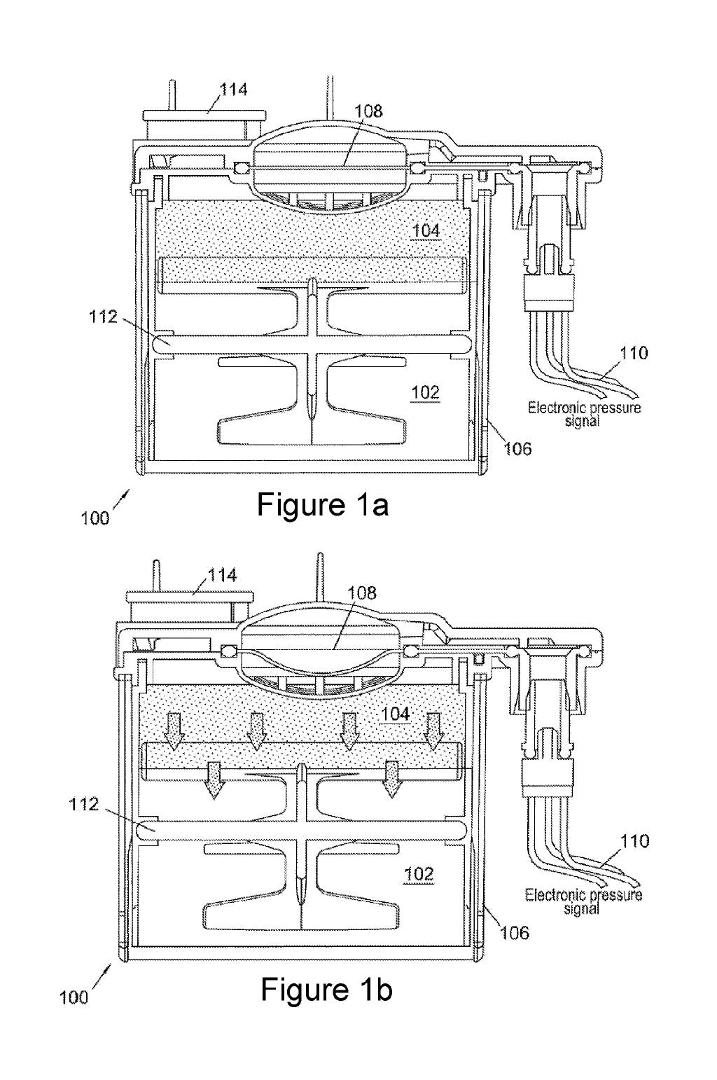

FIG. 2 shows an example of a test unit configured to accept a culture vessel of the type shown in FIG. 1;

FIG. 3 shows a block diagram of a test unit of the type shown in FIG. 2 configured to implement a test method according to an embodiment of the invention;

FIG. 4 shows a flow diagram of a procedure for operation of the test unit of FIG. 3;

FIG. 5 shows, schematically, a method of preparing a culture vessel containing growth media according to an embodiment of the invention;

FIGS. 6a and 6b show, respectively, example growth curves for E. Coli and a related graph of the number of E. Coli CFUs initially present in a sample and the time to detection of a threshold pressure change; and

FIG. 7 shows a block diagram of test software configured to test for regulatory compliance.

### DETAILED DESCRIPTION OF PREFERRED EMBODIMENTS

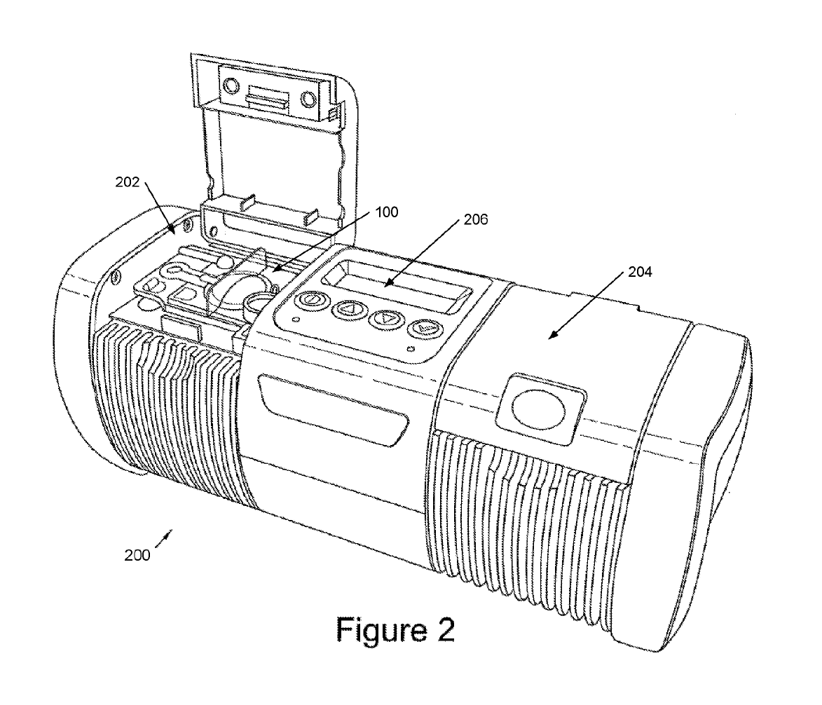

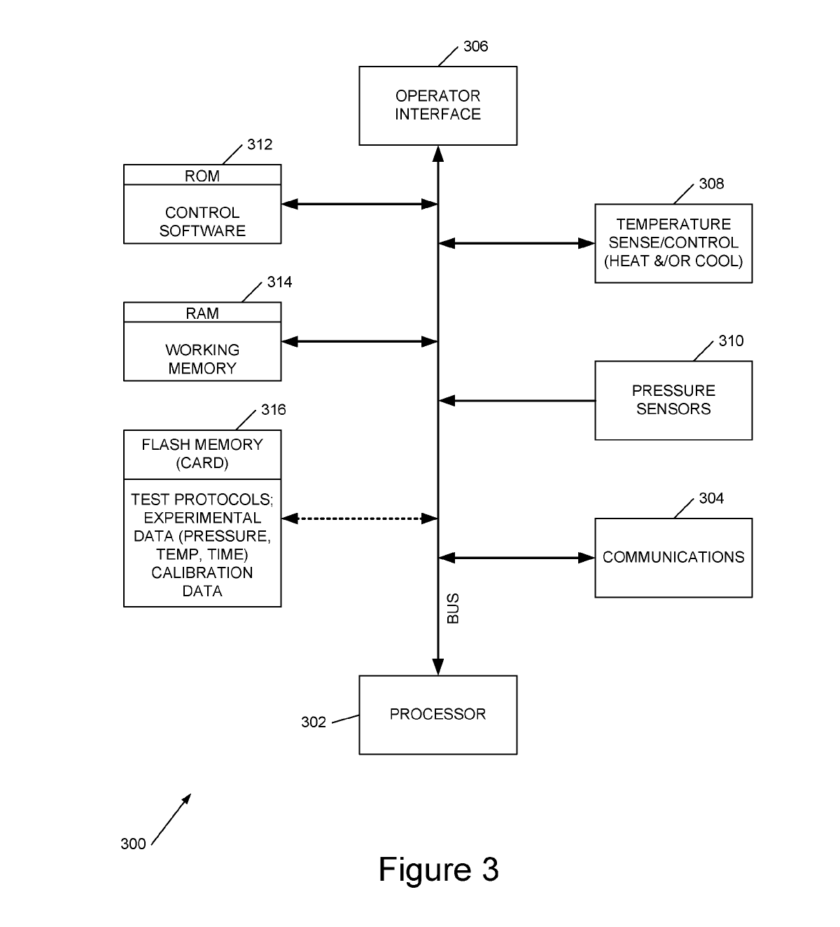

Referring now to FIG. 3, this shows a block diagram of a test unit 300 of the general type shown in FIG. 2, configured to implement an embodiment of a method according to the invention. The block diagram is simplified to facilitate understanding omitting, for example, safety features. Thus a processor 302 is coupled to one or more communications interfaces 304, such as a USB port and/or network connection, and to an operator interface 306, for example similar to interface 206 at FIG. 2. The processor is also coupled to a temperature control system 308, for each culture vessel. This comprises a temperature sensor and heater and/or cooler, operating under control of processor 302 according to control software stored in programmed memory 312. A pressure sensor 310 senses the pressure of the or each culture vessel, again for use by the control software. Optionally other sensors may be included, for example a colour sensor to sense a colour or colour change of a culture medium. In a simple embodiment this may comprise a wavelength-selective optical illumination source and/or a wavelength-selective detector, for example an optical detector with a colour filter. The system includes working memory 314 and non-volatile (in embodiments Flash™) memory 316, optionally on a removable card or the like. The non-volatile memory 316 stores data defining a set of test protocols, one for each species of bacteria to be detected, stores logged experimental data, in particular pressure, temperature, and time data. Where enumeration of the number of CFUs in the initial sample is desired, memory 316 also stores calibration data. The calibration data is preferably in the form of a look-up table including a set of rows, one for each of a different species or genus of bacteria in combination with a specific growth medium. Optionally the calibration data may also include a row for generic entity types (either any entity, or classes of entity such as bacteria, fungi and so forth) in combination with non-specific growth medium, where the unit is used for non-specific enumeration of organisms. The data in each row defines a straight line log N against time relationship (where N is an initial number of CFUs in the sample) for each species/genus, optionally but preferably including data which is useable to define a range for N, such as standard deviation data.

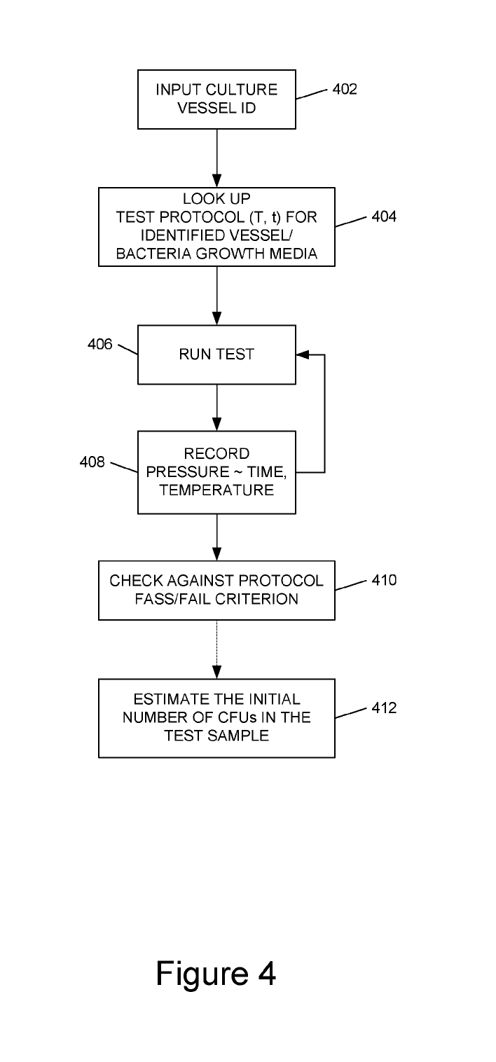

FIG. 4 shows a flow diagram of a procedure for operation of the test unit of FIG. 3. Thus at step 402 the procedure inputs an identifier for the culture vessel which identifies the species/genus of bacteria for which the growth medium is specially adapted. The procedure then looks up the corresponding test protocol. This defines a temperature or temperature profile for the test, and preferably the pressure detection point, that is a pre-set pressure change required for a positive detection of the specific target organism (step 404). The test is then run 406 and pressure and time and optionally temperature data is stored in memory 316. The test may be stopped when a pass/fail criterion, for example stored in the previously mentioned look-up table, is met. Alternatively a set of pressure-time data may be obtained and afterwards tested to determine whether or not the sample meets a pass/fail criterion (step 410). Optionally the initial number of CFUs in the test sample may be estimated 412, using the corresponding relationship stored in the look-up table, determining the estimated number from the set of pressure-time data or, more simply, from the time interval between starting the test and meeting the criterion at step 410.

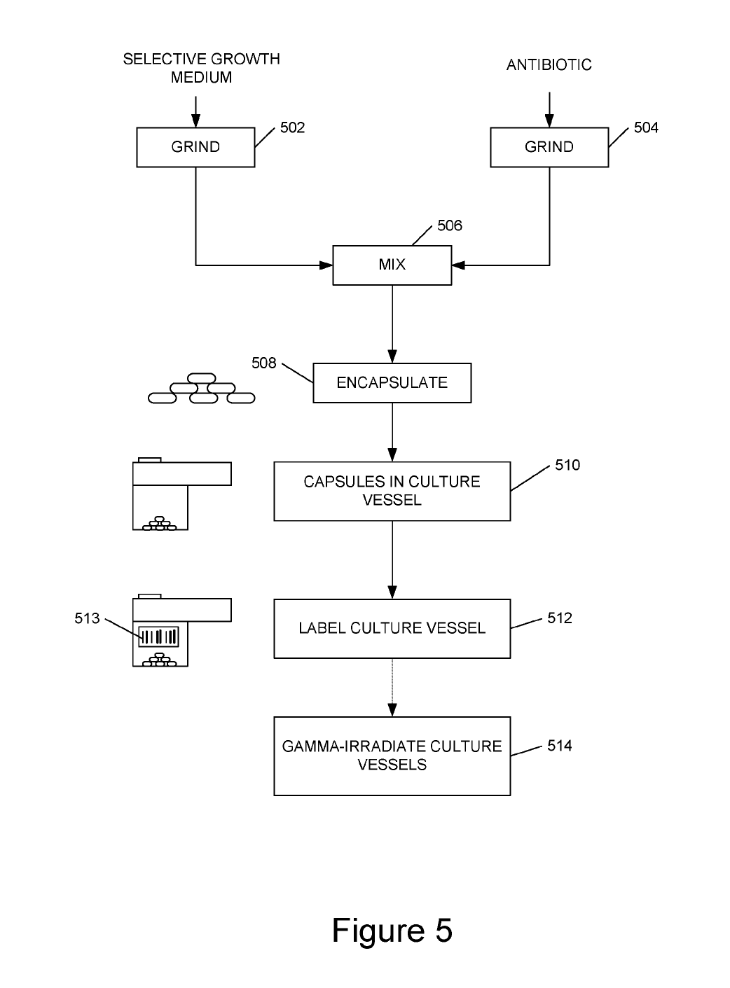

FIG. 5 shows, schematically, a method of preparing a culture vessel containing selective growth media according to an embodiment of the invention. Thus in embodiments selective growth medium and antibiotic are ground 502, 504 in a ball mill and then mixed 506 and encapsulated 508, for example in HPMC. Alternatively the starting materials may be ground together. Suitable selective growth media for a range of different bacteria in dried powder form and suitable antibiotics, can be obtained for example from Oxoid Limited UK. The skilled person will appreciate that many different types of culture media are known for different types of bacteria; in general these have some specificity. In principle any of these known types of culture media may be employed in embodiments of the invention. Similarly many different types of antibiotic are known, providing more or less selectivity, and again in principle any suitable type of antibiotic may be employed.

Once the capsules have been manufactured they are placed in the chamber of a culture vessel, preferably in a defined quantity (step 510) and the culture vessel is labelled (512) with an identifier 513, such as a bar code, corresponding to the bacteria (or other entity) towards which the capsules are targeted. It will be appreciated that at this point the growth medium or mixture in a capsule, whilst being targeted at a specific entity, may nonetheless be capable of growing other types of entity, for example different species of bacteria. The culture vessels are then sterilised by irradiation by gamma rays (514), for example at a dosage of 25-40 kGy, a service provided, for example, by Synergy Health, UK.

The labelled culture vessels may then be used in the procedure of FIG. 4. This provides additional selectivity via the selection of an appropriate test protocol corresponding to the identification of bacteria (or other entity) type on the culture vessel label. A combination of this selective protocol with the selective growth medium has been established to be sufficient to target the identified entity type, such that the results of the test protocol are effectively specific to a target.

One example of a selective growth medium which may be employed in a procedure as described above is a dry-form MacConkey Broth. This differentially selects for the growth of coliform organisms (coli-aerogenes—bacteria of the genera Escherichia and Aerobacter). More particularly this medium contains bile salts which inhibit most Gram-positive bacteria. The manufacture of this growth medium is well known to the skilled person; one formulation comprises approximately, for example: 20 g peptone, 10 g lactose, 5 g bile salts, 5 g sodium chloride, made up to 1 litre of water. Optionally an indicator may be added to monitor acid production, for example crystal violet (which also inhibits some Gram-positive bacteria) or bromocresol purple—the colour change (for example from purple to yellow) may be combined with a colorimetric protocol as mentioned previously. The skilled person will be aware that there are many variations on this basic formula, depending upon which inhibitory effects on growth are desirable and/or undesirable. Once the broth has been produced it may be dried and stored; alternatively it may be purchased in dehydrated form from suppliers such as Oxoid, LabM and Merck (trade marks).

Another example of a selective growth medium, selective for Salmonella (a Gram-negative bacteria) such as Salmonella spp., may be produced as described below: An example growth medium formulation comprises approximately: 5 g peptone, 5 g yeast extract, 5 g salt buffer, and optional additional growth promoter, made up to 1 litre of water. Again such a growth medium may be purchased in dehydrated form from suppliers such as Oxoid, LabM and Merck (trade marks). To this growth medium is preferably also added an antibiotic such as novobiocin (an inhibitor for Proteus) and/or cefsulodin (an inhibitor for Pseudomonas). Rather than add antibiotic to the broth, the antibiotic is added in dried form—both the antibiotic and the (dry) growth medium are ground (in a ball mill) and combined as a single dehydrated material within water-soluble capsules, which are after gamma-irradiated. Again, optionally, one or more chromogen indicators may also be added to the growth medium if necessary, for example, chromogens which react to the presence of specific enzymes by changing colour (these are again available to purchase).

With these approaches the selective growth medium is kept sterile and automatically rehydrates on use (when an aqueous sample is added to the culture chamber), without the need for a user to handle potentially hazardous materials.

The skilled person will appreciate that many other variations of dry selective growth medium may be produced by similar techniques, for example modifying existing formulations for the production of agar plates.

As an example of a test protocol to detect Salmonella the above-described selective growth medium for Salmonella, including both novobiocin and cefsulodin, is maintained at a temperature of between 36° C. and 37° C. (for 48 hours). Outside this range the selective, differential growth of, in this example, Salmonella is substantially less effective. As an example of a test protocol to detect E. Coli, the above-described E. Coli-selective growth medium was used at either 36° C.+/−1° C. for general coliforms or at 44° C.+/−1° C. for detecting E.Coli only (for 48 hours), depending on the type of liquid sample employed: In this example the liquid was a food stuff with two different flavourings—the test protocol was determined by a series of experiments to establish which was most reliable and effective; the protocol used was found to depend on the flavouring (and presumed composition) of the foodstuff. The skilled person will recognise that other test protocols may be established in a similar manner. Without wishing to be bound by theory, broadly speaking the optimum temperature appears to be the maximum temperature at which the target organism survives and reproduces rapidly, thus inhibiting other organisms from growing where this temperature is too high for them. However other effects may also be operating. In general it is not necessary to employ a time cut-off or detection-time window for a protocol, although in principle one could be employed to provide a differential between slower and faster growing organisms and/or between samples with different initial numbers of CFUs. As noted above, a colour change of the test sample may in principle be used to provide an additional differential detection parameter—for example, detection of a colour change is an important part of an E.Coli or coliform test. Such a test may in principle be used either as a further target entity detection parameter or, perhaps more usefully, as an indication of potential reliability of a test (for example indicating “contamination” in the sense that good differentiation of the target organism has not been achieved).

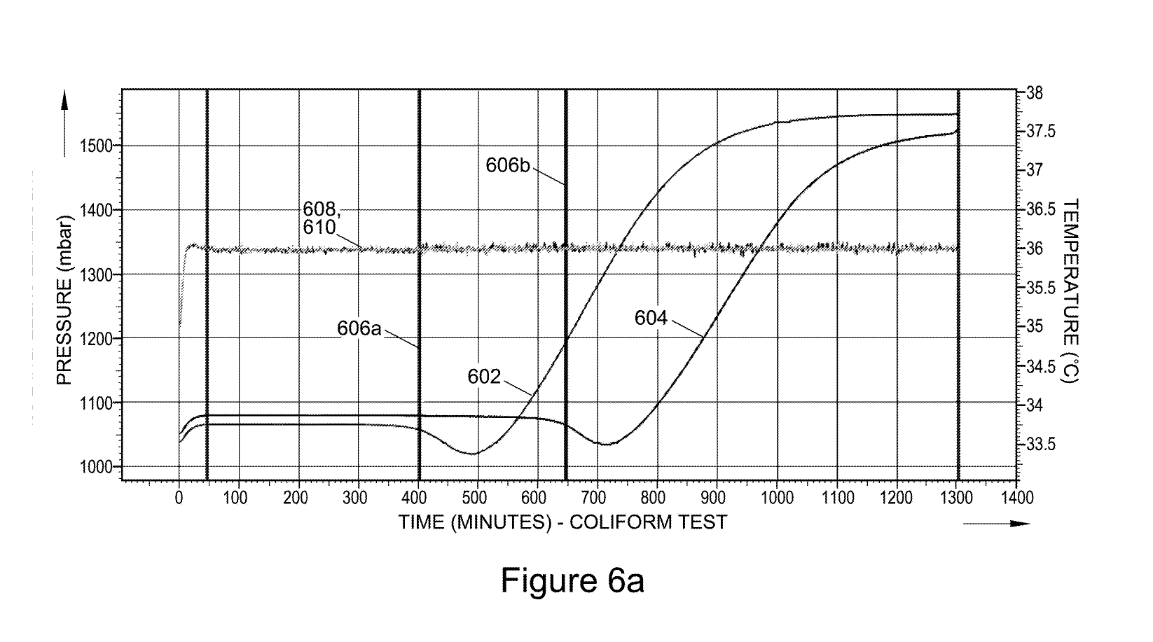

Referring now to FIG. 6a, this shows two example growth curves 602, 604 for E.Coli, showing pressure against time over approximately a 24 hour period. The sample of curve 602 had an initial number of 1000 CFUs in the sample; the sample of curve 604 had an initial number of 30 CFUs in the sample. The vertical lines 606a,b mark the respective times to detection of a threshold pressure change (the same threshold pressure change for both samples). As can be seen in this example, the presence of E.Coli is detected by an initial drop in pressure prior to a subsequent rise in pressure. Lines 608, 610 show the (steady, regulated) temperature of the samples, in this case in a test protocol of 36° C.+/−1° C. (although as can be seen, the temperature is preferably controlled to better than+/−0.5° C.,+/−0.25° C. or+/−0.1° C.).

The threshold change in pressure for detection of an organism may be either positive or negative (or even a combination of the two, for example a defined fall followed by a defined rise, or vice-versa). The particular shape of the growth curve of FIG. 6a is by way of example—other bacteria can have different shaped curves. For example, E. Coli is a facultative anaerobe, that is it can grow wither aerobically or anaerobically, although has a preference for the former as this enables the organism to generate more energy faster. Thus in the example of FIG. 6a the pressure curve initially drops, whilst oxygen is being used, and then the bacteria switch to anaerobic metabolism and begin producing carbon dioxide (a product of this fermentation process), so that the pressure starts to rise again.

Many experiments have also been performed to measure time to detection (of an organism) against the number of organisms initially present, using a specific growth medium and the corresponding test protocol. In every case, a good correlation is seen between the logarithm (to base 10, for example) of the initial number of CFUs/gram or CFUs/ml in the test liquid sample and the time to detection of the organism (a straight line with a negative slope: the smaller the initial number of organisms the longer the time to detection). This relationship has held over a wide range, for example from <10 CFUs/g to>1000 CFUs/g.

Referring to FIG. 6b, this shows an example of a graph of the number of E.Coli CFUs initially present in a sample and the time to detection of a threshold pressure change, for a range of different initial CFU inoculations (derived by serial dilution). As can be seen there is a high degree of correlation present, even for low initial CFU values. Moreover, because of the sensitivity of the pressure change measurement the time to detection can be seen to be very quick. It can be appreciated that once a curve such as that shown in FIG. 6b has been measured, and one or more parameters defining the curve stored, it is straightforward to use the curve to read off a predicted initial number of bacteria in a sample given the time to detection.

One application for a system as described above is establishing regulatory compliance, for example of a foodstuff or for waste water discharge. By way of example the case of a ballast water treatment system for a ship will be considered—but the principles apply in a similar way to other applications. In this example a ship owner needs to be confident that waste water discharged satisfies a regulation defining the concentrations of one or more species bacteria permitted in the discharge, for example maximum concentrations of E.Coli, Enterococci and Cholera (for example no more than 250 CFU of E.Coli in 100 ml of treated water). Further there may be multiple different regulations to comply with, in this example depending on the location of the ship.

In addition the coast guard needs to be able to verify that the regulatory requirement(s) have been met, so that there is an auditing requirement, for the results and for the data on which they are based. This latter requirement (where present) can be addressed by encrypting at least the data from which the test results were derived, and by ensuring that only encrypted data are permanently stored within the device (to avoid modification). The decryption key may be built securely into the test machine/computer system (in such a way that it is not easily accessible by decompiling the program code).

To address the regulatory requirements, a one-to-one relationship is established between a calibration and a test protocol, and the protocol is fixed once the calibration procedure has begun. Many tests make up a single calibration; preferably the system software enables a user to create a new calibration by selecting a test and a protocol that will be used to generate the calibration data. The calibration is represented by a parameter(s) of a graph of the data used to create it; many tests may be validated against a single calibration, and these tests must use the protocol that is associated with the calibration. A preferred output from a test comprises an indication of the number of CFUs initially present in the sample and/or a pass/fail indication (when linked to a regulation).

An example calibration procedure follows. Preferably a user is taken though such a procedure step by step. Thus for a sample (of water) a series of n experiments is conducted, each with a different, known initial concentration of CFU/ml of a target bacteria; preferably n=>5. Preferably the range of CFU/ml should be appropriate to the expected range in the samples. The results can then be plotted (for visualisation), and a curve is fitted by any standard means. The calibration may then optionally be verified by checking an unknown sample against another test technique. In the case of salt water, optionally salt concentration may be varied—if an effect is seen a series of calibration curves may be determined for a range of different salt concentrations. The same principle may be applied to other sample-characterising variables, for example a flavour/composition characterising variable in the case of food or drink. A calibration procedure of this type is followed for each separate target entity it is desired to evaluate.

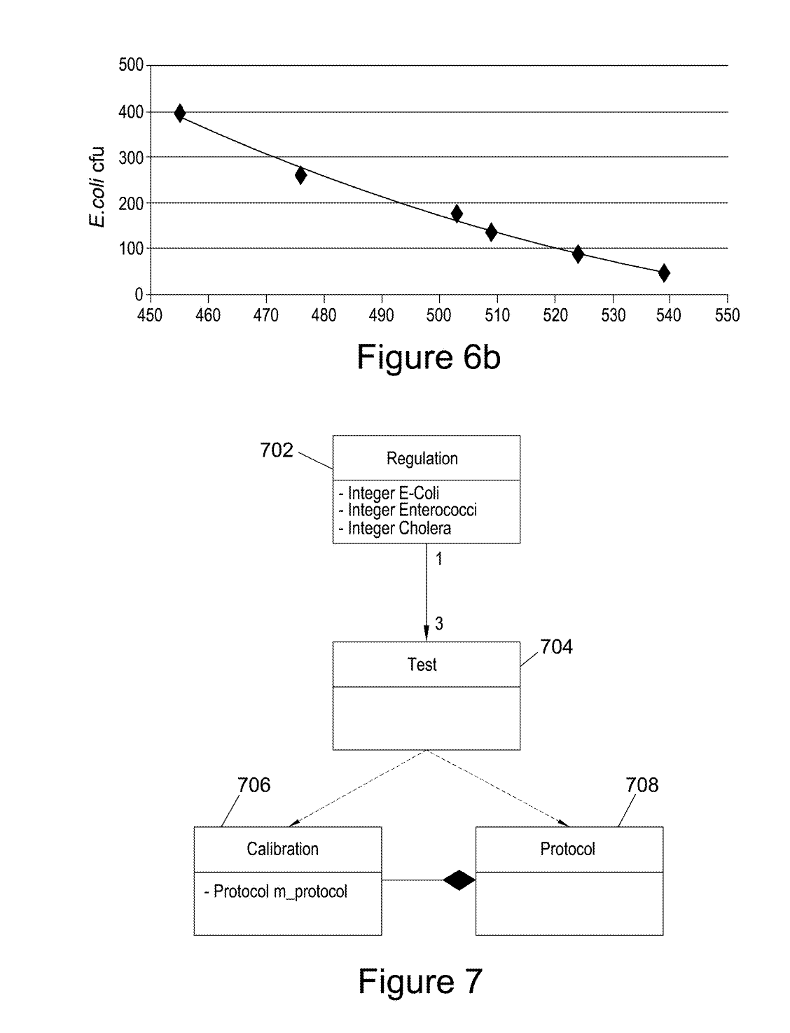

In preferred embodiments of the software provision is made to create a “regulation”, in embodiments a software object, to facilitate establishing conformance with a regulation. Typically such a regulation object links to, or is associated with, one or more tests for one or more specific, target biological entities which are the subject of the regulation. The tests each define a permitted maximum level of the entity in the sample, for example as CFU/ml. In embodiments a regulation does not have a direct relationship with a calibration, but there is an indirect relationship. Thus referring to FIG. 7, regulation object 702 defines maximum permitted concentrations of E.Coli, Enterococci and Cholera, and provides concentration limit data to three tests 704, one for each of the targets, used to define respective pass/fail criteria for the tests. Each test 704 links to respective calibration data 706 and to a dedicate test protocol 708, applied as described previously. In this way the system is able to perform the tests, log the data (preferably in encrypted form), and provide a simple pass/fail output for the regulation as well as, optionally, for each test separately.

No doubt many other effective alternatives will occur to the skilled person. It will be understood that the invention is not limited to the described embodiments and encompasses modifications apparent to those skilled in the art lying within the spirit and scope of the claims appended hereto.

1. A method of selectively testing a liquid sample for the presence of a specific living biological entity, the method comprising:
preparing a plurality of culture vessels each for selective detection of the presence of a different living biological entity, wherein preparing a said culture vessel comprises:
providing a culture vessel comprising a sealable chamber for receiving and culturing a liquid sample such that presence of said biological entity is detectable by detecting a change in pressure in a headspace of said chamber;
providing a selective growth medium in said culture vessel, wherein said selective growth medium is in a dry form and selectively facilitates growth of a specific living biological entity, and
providing the culture vessel with an identifier to identity said specific biological entity;
providing a test system comprising a device to receive said culture vessel and to detect said change in pressure in said headspace of said chamber, the device further comprising a temperature control system, and a data processor to monitor said detected change in pressure, to control said temperature control system to regulate a temperature of a liquid sample in said culture vessel, and to signal detection of the presence of a biological entity in said culture vessel from said detected change in pressure;
storing a plurality of entity-selective test protocols in a memory of said test system, wherein a said test protocol defines one or both of a temperature of incubation of said liquid sample in said culture vessel and a threshold change in pressure in said headspace of said chamber for detection of the target entity, wherein said test protocols are respectively identified according to one of a plurality of specific biological entities for which they selectively facilitate growth;
selecting, from said plurality of culture vessels, a culture vessel to selectively detect a target said specific biological entity;
providing a test liquid sample to said chamber of said selected culture vessel;
using the identifier on said selected culture vessel to select a corresponding test protocol for said target specific biological entity; and
running said selected test protocol on said test system to incubate said test liquid sample according to said test protocol to determine whether said target specific biological entity is present within said test liquid sample;
wherein said determination comprises selection dependent upon both said selective growth medium and said entity-selective test protocol.

2. A method as claimed in claim 1 wherein said test protocol defines both said temperature of incubation of said liquid sample in said culture vessel and said threshold change in pressure in said headspace of said chamber for detection of the target entity.

3. A method as claimed in claim 1 wherein said dry-form selective growth medium is encapsulated within water-soluble capsules to preserve said selective growth medium and provide said selective growth medium for growth of said entities by rehydration of said selective growth medium on addition of said liquid sample.

4. A method as claimed in claim 3 wherein said preparing of said culture vessel further comprises combining said selective growth medium with a selective growth inhibiting antibiotic, to selectively inhibit growth of living biological entities other than said specific living biological entity.

5. A method as claimed in claim 4 wherein said combining comprises combining particulates of said dry-form selective growth medium and particulates of said selective growth inhibiting substance and encapsulating said combined particulates within water-soluble capsule housings, to provide said combined selective growth medium and selective growth inhibiting substance in the form of water-soluble capsules within said culture vessel for rehydration on addition of said liquid sample.

6. A method as claimed in claim 1 further comprising irradiating said combined selective growth medium, and where present said selective growth inhibiting substance, within said culture vessel with gamma rays.

7. A method as claimed in claim 1 further comprising:
storing, in said memory of said test system, calibration data defining a relationship between a time to a threshold said change in pressure and an initial number of said biological entities; and
determining an estimated number of said target specific biological entities in said test liquid sample from said detected change in pressure and said calibration data.

8. A liquid sample test system for selectively testing a liquid sample for the presence of a specific living biological entity, the system comprising:
a plurality of culture vessels each for selective detection of the presence of a different living biological entity, a said culture vessel comprising a sealable chamber for receiving and culturing a liquid sample such that presence of said biological entity is detectable by detecting a change in pressure in a headspace of said chamber, said culture vessel chamber including a selective growth medium in said culture vessel, wherein said selective growth medium is in a dry form and selectively facilitates growth of a specific living biological entity, said culture vessel having an identifier to identify said specific biological entity;
a test system comprising a device to receive said culture vessel and to detect a change in pressure in said headspace of said chamber, the device further comprising a temperature control system, and a data processor to monitor said detected change in pressure, to control said temperature control system to regulate a temperature of a liquid sample in said culture vessel, and to signal detection of the presence of a biological entity in said culture vessel from said detected change in pressure;
wherein said data processor comprises memory storing a plurality of entity-selective test protocols, wherein a said test protocol defines one or both of a temperature of incubation of said liquid sample in said culture vessel and a threshold change in pressure in said headspace of said chamber for detection of the target entity, wherein said test protocols are respectively identified according to one of a plurality of specific biological entities for which they selectively facilitate growth; and
wherein the identifier on said selected culture vessel is usable to select a corresponding test protocol for said target specific biological entity; and
wherein said selected test protocol is useable by said test system to incubate said test liquid sample according to said test protocol to determine whether said target specific biological entity is present within a test liquid sample.

9. A system as claimed in claim 8 wherein said selective growth medium is combined with a selective growth inhibiting substance to selectively inhibit growth of living biological entities other than said specific living biological entity.

10. A system as claimed in claim 9 wherein said memory further stores calibration data defining a relationship between a time to a threshold said change in pressure and an initial number of said biological entities, and wherein said data processor comprises processor control code to determine an estimated number of said target specific biological entities in said test liquid sample from said detected change in pressure and said calibration data.

11. A method of preparing a culture vessel, in particular for the method of claim 1, for selectively testing a liquid sample for the presence of a specific living biological entity, the method comprising:
providing a culture vessel comprising a sealable chamber for receiving and culturing a liquid sample such that presence of said biological entity is detectable by detecting a change in pressure in a headspace of said chamber;
providing a selective growth medium in said culture vessel, wherein said selective growth medium is in a dry form and selectively facilitates growth of a specific living biological entity; and
providing the culture vessel with an identifier to identity said specific biological entity.

12. A method as claimed in claim 11 further comprising combining said selective growth medium with a selective growth inhibiting substance to selectively inhibit growth of living biological entities other than said specific living biological entity.

13. A method as claimed in claim 12 wherein said combining comprises combining particulates of said dry-form selective growth medium and particulates of said selective growth inhibiting substance, and encapsulating said combined particulates within water-soluble capsule housings to provide said combined selective growth medium and selective growth inhibiting substance in the form of water-soluble capsules within said culture vessel, for preservation of said growth medium and inhibiting substance and rehydration of said growth medium and inhibiting substance by a water-based said liquid sample.

14. A method as claimed in claim 12 wherein said selective growth inhibiting substance comprises an antibiotic.

15. A method as claimed in claim 12, further comprising irradiating said combined selective growth medium, and where present said selective growth inhibiting substance, within said culture vessel with gamma rays.

16-22. (canceled)

23. A liquid sample test system for testing a liquid sample for the presence of a living biological entity, the system comprising:
a culture vessel comprising a sealable chamber for receiving and culturing a liquid sample such that presence of said biological entity is detectable by detecting a change in pressure in a headspace of said chamber, said culture vessel chamber including a growth medium for said living biological entity; and
a test system comprising a device to receive said culture vessel and to detect said change in pressure of said headspace of said chamber, the device further comprising a temperature control system, and a data processor to monitor said detected change in pressure, to control said temperature control system to regulate a temperature of a liquid sample in said culture vessel, and to signal detection of the presence of a biological entity in said culture vessel from said detected change in pressure;
wherein said data processor comprises memory storing calibration data defining a relationship between a time to a threshold said change in pressure and an initial number of said biological entities; and
wherein said data processor comprises processor control code to determine an estimated number of said target specific biological entities in said test liquid sample from said detected change in pressure and said calibration data.

24. A liquid sample test system as claimed in claim 23 wherein said code further comprises code to determine a numeric range for said estimated number dependent on statistical data for said defined relationship stored in said memory.

25. A liquid sample test system as claimed in claim 23 wherein said calibration data defines a different said relationship for each of a plurality of different types of said biological data, and wherein said code is configured to determine said estimated number or range dependent on input data defining a said type of biological entity and the corresponding said relationship.

26. (canceled)

27. A liquid sample test system as claimed in claim 23, wherein said dry-form selective growth medium is encapsulated within water-soluble capsules, combined with a selective growth inhibiting substance, for preservation and rehydration by a water-based said liquid sample to supply said growth medium when said liquid sample is added.

28-34. (canceled)

35. A method of selectively testing a liquid sample for the presence of a living biological entity, the method comprising:
providing a culture vessel comprising a sealable chamber for receiving and culturing a liquid sample such that presence of said biological entity is detectable by detecting a change in pressure in a headspace of said chamber;
providing a growth medium in said culture vessel, wherein said growth medium is in a dry form and facilitates growth of a living biological entity, and
providing a test system comprising a device to receive said culture vessel and to detect said change in pressure in said headspace of said chamber, the device further comprising a temperature control system, and a data processor to monitor said detected change in pressure, to control said temperature control system to regulate a temperature of a liquid sample in said culture vessel, and to signal detection of the presence of a biological entity in said culture vessel from said detected change in pressure;
providing a test liquid sample to said chamber of said selected culture vessel;
incubating said test liquid sample to determine whether said target specific biological entity is present within said test liquid sample; and then
rendering said culture vessel non-viable or non-culturable by raising a temperature of said incubated test liquid sample to greater than a threshold temperature for greater than a threshold time, and wherein said incubated test liquid sample is sealed within said culture vessel for disposal after said treating.

36-28. (canceled)
